(19) United States
(12) Patent Application Publication
(10) Pub. No.: US 2010/0099710 A1
(43) Pub. Date: Apr. 22, 2010

(54) SRC FAMILY KINASE INHIBITORS

(76) Inventors: Lene Jensen, Malov (DK); Jef Fensholdt, Stenlose (DK)

Correspondence Address: BIRCH STEWART KOLASCH & BIRCH, PO BOX 747, FALLS CHURCH, VA, 22040-0747, US

(21) Appl. No.: 12/596,636

(22) PCT Filed: Apr. 16, 2008
(86) PCT No.: PCT/DK08/00134

§ 371 Date: Oct. 19, 2009

Related U.S. Application Data
(60) Provisional application No. 60/912,792, filed on Apr. 19, 2007.

(51) Int. Cl. A61K 31/4545; C12N 5/071; C07D 213/44; C07D 211/72; C07D 211/68; A61K 31/444; A61K 31/44; A61P 31/00

(52) U.S. Cl. 514/318; 435/325; 546/262; 546/304; 546/193; 514/350; 514/332

(57) ABSTRACT

The invention relates to compounds of general formula (I) wherein X, A, R₁ and R₂ are as defined herein for use as antiinflammatory agents capable of modulating the activity of a protein tyrosine kinase of the Src family.

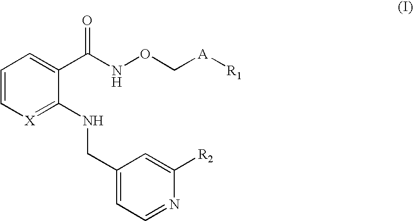

### FIELD OF INVENTION

The present invention relates to compounds of formula I as defined below for use as inhibitors of protein tyrosine kinases of the Src kinase family as well as a method of prevention or treatment of inflammatory diseases or disorders involving protein tyrosine kinases of the Src kinase family.

### BACKGROUND OF THE INVENTION

Protein tyrosine kinases are a family of enzymes catalysing the transfer of the terminal phosphate of adenosine triphosphate to tyrosine residues in protein substrates. Phosphorylation of tyrosine residues on protein substrates leads to transduction of intracellular signals which regulate a wide variety of intracellular processes such as growth and activation of cells of the immune system, e.g. T-cells. As T-cell activation is implicated in a number of inflammatory conditions and other disorders of the immune system (e.g. autoimmune diseases), modulation of the activity of protein tyrosine kinases appears to be an attractive route to the management of inflammatory diseases. A large number of protein tyrosine kinases have been identified which may be receptor protein tyrosine kinases, e.g. the insulin receptor, or non-receptor protein tyrosine kinases.

Protein tyrosine kinases of the Src family have been found to be particularly important for intracellular signal transduction related to inflammatory responses (cf. D. Okutani et al., Am. J. Physiol. Lung Cell Mol. Physiol. 291, 2006, pp. L129-L141; C. A. Lowell, Mol. Immunol. 41, 2004, pp. 631-643). While some of Src family protein tyrosine kinases, e.g. Src, Yes and Fyn, are expressed in a variety of cell types and tissues, the expression of others is restricted to specific cell types, e.g. hematopoietic cells. Thus, the protein tyrosine kinase Lck is expressed almost exclusively in T-cells as the first signalling molecule to be activated downstream of the T-cell receptor, and its activity is essential for T-cell signal transduction. Expression of Hck, Lyn and Fgr is increased by inflammatory stimuli such as LPS in mature monocytes and macrophages. Also, if gene expression of the main B-cell Src family kinases, namely Lyn, Fyn and Blk, is disrupted, immature B-cells are prevented from developing into mature B-cells. Src family kinases have also been identified as essential for the recruitment and activation of monocytes, macrophages and neutrophils as well as being involved in the inflammatory response of tissue cells. For example, it has been found that expression of Hck, Lyn and Fgr is increased by inflammatory stimuli such as LPS in mature monocytes and macrophages.

A substantial number of autoimmune and inflammatory diseases involve the activation of T-cells and B-cells as well as other cells of the immune system such as monocytes and macrophages. Compounds which are capable of inhibiting activation of these cell types are therefore regarded as useful therapeutic agents in the treatment of such diseases.

In WO 2005/054179, compounds of formula I are disclosed as VEGF receptor tyrosine kinase inhibitors and proposed for the treatment of diseases associated with VEGF-dependent angiogenesis and cell proliferation. There is no disclosure in this document that the compounds might be active as inhibitors of other kinases such as protein tyrosine kinases of the Src family and as such show utility as antiinflammatory and immunomodulating agents.

### SUMMARY OF THE INVENTION

In the course of research leading to the present invention, it has surprisingly be found that a subset of compounds disclosed in WO 2005/054179 has activity on a number of other tyrosine kinases as well, such as protein tyrosine kinases involved in inflammatory or immune response in cells. Thus, in addition to acting as inhibitors of the VEGF receptor, the compounds are capable of modulating the activity of protein tyrosine kinases of the Src family which, as indicated above, are upregulated in many non-infectious inflammatory or autoimmune diseases and disorders.

Accordingly, the present invention relates to a compound of general formula I

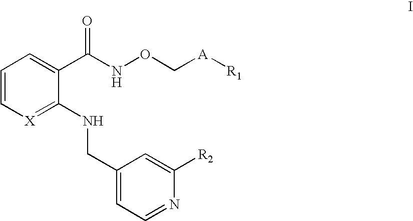

wherein X represents nitrogen or CH;
A represents a straight, branched and/or cyclic, saturated or unsaturated hydrocarbon radical, a heterocycloalkyl, a heterocycloalkenyl, or a heteroaryl, all of which are optionally substituted with one or more substituents independently selected from the group consisting of R₁;
R₁ represents oxo, halogen, trifluoromethyl, hydroxyl, amino, nitro, carboxy, cyano, alkoxy, alkylthio, alkoxycarbonyl, alkylcarbonyloxy, alkoxycarbonyloxy, alkylureido, alkylthioureido, alkylcarbonyl, alkoxysulfonyloxy, aminosulfonyl, arylsulfonyl, alkylsulfonylamino, arylsulfonylamino, heteroarylsulfonylamino, alkylsulfonyl, formyl, aminocarbonyl, alkylcarbonylamino, alkylaminocarbonyl, aminocarbonyloxy, heterocycloalkyl, heterocycloalkenyl, heteroaryl and a straight or branched, saturated or unsaturated hydrocarbon radical, wherein said amino, alkoxy, alkylthio, alkoxycarbonyl, alkylcarbonyloxy, alkoxycarbonyloxy, alkylureido, alkylthioureido, alkylcarbonyl, alkoxysulfonyloxy, aminosulfonyl, arylsulfonyl, alkylsulfonylamino, arylsulfonylamino, heteroarylsulfonylamino, alkylsulfonyl, aminocarbonyl, alkylcarbonylamino, alkylaminocarbonyl, aminocarbonyloxy, heterocycloalkyl, heterocycloalkenyl, heteroaryl and straight or branched, saturated or unsaturated hydrocarbon radical are optionally substituted by one or more substituents independently selected from the group consisting of R₃;
R₂ represents amino, aminosulfonyl, aminocarbonyl, alkylureido, alkylthioureido or aminocarbonyloxy, wherein each amino, aminosulfonyl, aminocarbonyl, alkylureido, alkylthioureido or aminocarbonyloxy is optionally substituted with one or more substituents independently selected from the group consisting of R₃;
R₃ represents hydrogen, cycloalkyl, alkyl, aryl, heteroaryl, heterocycloalkyl, heterocycloalkenyl, heterocycloalkyl-heteroaryl, heterocycloalkylcarbonylamino, cycloalkenyl, alkenyl, alkynyl, alkoxy, alkoxyimino, alkylthio, alkoxycarbonyl, alkylcarbonyloxy, alkenylcarbonyloxy, alkoxycarbonyloxy, alkylureido, alkylthioureido, alkylcarbonyl, alkoxysulfonyloxy, aminosulfonyl, alkylsulfonylamino, alkylsulfonyl, arylsulfonyl, formyl, aminocarbonyl, and alkylcarbonylamino, wherein said amino, imino, cycloalkyl, alkyl, aryl, heteroaryl, heterocycloalkyl, heterocycloalkenyl, heterocycloalkyl-heteroaryl, heterocycloalkylcarbonylamino, cycloalkenyl, alkenyl, alkynyl, alkoxy, alkoxyimino, alkylthio, alkoxycarbonyl, alkylcarbonyloxy, alkenylcarbonyloxy, alkoxycarbonyloxy, alkylureido, alkylthioureido, alkylcarbonyl, alkoxysulfonyloxy, aminosulfonyl, alkylsulfonylamino, alkylsulfonyl, arylsulfonyl, aminocarbonyl, and alkylcarbonylamino are optionally substituted by one or more substituents independently selected from the group consisting of hydrogen, halogen, oxo, thioxo, hydroxyl, amino, imino, nitro, carboxy, cyano, alkoxy, alkylthio, alkoxycarbonyl, alkylcarbonyloxy, alkoxycarbonyloxy, alkylcarbonyl, alkoxysulfonyloxy, aminosulfonyl, alkylsulfonylamino, alkylsulfonyl, arylsulfonyl, aminocarbonyloxy, heteroarylsulfonylamino, formyl, aminocarbonyl, trifluoromethyl, alkylcarbonylamino, heterocycloalkyl, heterocycloalkenyl, aryl, alkylureido, alkylthioureido, heteroaryl, cycloalkyl, alkyl, cycloalkenyl, alkenyl, alkynyl, and alkylaminocarbonyl;
and pharmaceutically acceptable salts, hydrates, or solvates thereof;
for use as an antiiflammatory agent capable of modulating the activity of a protein tyrosine kinase of the Src family of protein tyrosine kinases.

In another aspect, the invention relates to a method of modulating the activity of a protein tyrosine kinase of the Src family of protein tyrosine kinases involved in inflammation and/or immune response in cells, the method comprising contacting a cell expressing at least one protein tyrosine kinase of the Src family of protein tyrosine kinases with a compound of formula I as defined above in an amount effective to modulate the activity of said protein tyrosine kinase in said cell.

In another aspect, the invention relates to a method of modulating the activity of a protein tyrosine kinase of the JAK family of protein tyrosine kinases involved in inflammation and/or immune response in cells, the method comprising contacting a cell expressing at least one protein tyrosine kinase of the JAK family of protein tyrosine kinases with a compound of formula I as defined above in an amount effective to modulate the activity of said protein tyrosine kinase in said cell.

In another aspect, the invention relates to a method of modulating the activity of serine/threonine kinase of the RAF family involved in inflammation and/or immune response in cells, the method comprising contacting a cell expressing a RAF family kinase with a compound of formula I as defined above in an amount effective to modulate the activity of said RAF family kinase in said cell.

In another aspect, the invention relates to a method of modulating the activity of a receptor tyrosine kinase selected from the group consisting of cKit and Fms/CSF-1R involved in inflammation and/or immune response in cells, the method comprising contacting a cell expressing at least one of cKit or Fms/CSF-1R with a compound of formula I as defined above in an amount effective to modulate the activity of said receptor tyrosine kinase in said cell.

In a further aspect, the invention relates to a method of reducing the proinflammatory activity in cells of a protein tyrosine kinase of the Src family of protein tyrosine kinases and/or the Jak-2 and/or Raf-1 and/or cKit and/or Fms/CSF-1R kinase, the method comprising contacting a cell expressing at least one protein tyrosine kinase of the Src family and/or the Jak-2 and/or Raf-1 and/or cKit and/or Fms/CSF-1R kinase with a compound of general formula I as defined above in an amount effective to inhibit the activity of said kinase.

In a still further aspect, the invention relates to the use of a compound of general formula I as defined above for the preparation of a pharmaceutical composition for the prevention or treatment of a non-infectious inflammatory or autoimmune disease or condition in which at least one protein tyrosine kinase of the Src family of protein tyrosine kinases and/or the Jak-2 and/or Raf-1 and/or cKit and/or Fms/CSF-1R kinase is significantly involved.

In a still further aspect, the invention relates to a method of preventing or treating a non-infectious inflammatory or autoimmune disease or condition in which at least one protein tyrosine kinase of the Src family of protein tyrosine kinases and/or the Jak-2 and/or Raf-1 and/or cKit and/or Fms/CSF-1R kinases is significantly involved, the method comprising administering, to a patient in need thereof, an effective amount of a compound of general formula I as defined above.

### DETAILED DESCRIPTION OF THE INVENTION

### Definitions

The term “hydrocarbon radical” is intended to indicate a radical containing only hydrogen and carbon atoms, it may contain one or more double and/or triple carbon-carbon bonds, and it may comprise cyclic moieties in combination with branched or linear moieties. Said hydrocarbon comprises 1-20 carbon atoms, and preferably comprises 1-12, e.g. 1-6, e.g. 1-4, e.g. 1-3, e.g. 1-2 carbon atoms. The term includes alkyl, alkenyl, cycloalkyl, cycloalkenyl, alkynyl and aryl, as indicated below.

In the present context, the term “alkyl” is intended to indicate the radical obtained when one hydrogen atom is removed from a hydrocarbon. Said alkyl comprises 1-20, preferably 1-12, such as 2-6, such as 3-4 carbon atoms. The term includes the subclasses normal alkyl(n-alkyl), secondary and tertiary alkyl, such as methyl, ethyl, n-propyl, isopropyl, n-butyl, isobutyl, sec.-butyl, tent.-butyl, pentyl, isopentyl, hexyl and isohexyl.

The term “cycloalkyl” is intended to indicate a saturated cycloalkane radical, including polycyclic radicals, such as bicyclic or tricyclic radicals, comprising 3-20 carbon atoms, preferably 3-10 carbon atoms, in particular 3-8 carbon atoms, such as 3-6 carbon atoms, such as 4-5 carbon atoms, e.g. cyclopropyl, cyclobutyl, cyclopentyl, cyclohexyl, cycloheptyl, cyclooctyl, bicyclo[2.2.1]heptyl and adamantyl.

The term “cycloalkenyl” is intended to indicate mono-, di- tri- or tetraunsaturated non-aromatic cyclic hydrocarbonsradicals, including polycyclic radicals, comprising 3-20 carbon atoms, typically comprising 3-10 carbon atoms, such as 3-6 carbon atoms, such as 4-5-carbon atoms, e.g. cyclopropenyl, cyclobutenyl, cyclopentenyl, cyclohexenyl, bicyclo[2.2.1]heptenyl, or bicyclo[4.1.0]heptenyl.

The term “alkenyl” is intended to indicate a mono-, di-, tri-, tetra- or pentaunsaturated hydrocarbon radical comprising 2-10 carbon atoms, in particular 2-6 carbon atoms, such as 2-4 carbon atoms, e.g. ethenyl, allyl, propenyl, butenyl, pentenyl, nonenyl, or hexenyl.

The term “alkynyl” is intended to indicate an hydrocarbon radical comprising 1-5 C—C triple bonds and 2-20 carbon atoms, the alkane chain typically comprising 2-10 carbon atoms, in particular 2-6 carbon atoms, such as 2-4 carbon atoms, e.g. ethynyl, propynyl, butynyl, pentynyl or hexynyl.

The term “heteroaryl” is intended to include radicals of heterocyclic aromatic rings, optionally fused with carbocyclic rings or heterocyclic rings, comprising 1-6 heteroatoms (selected from O, S and N) and 1-20 carbon atoms, such as 1-5 heteroatoms and 1-10 carbon atoms, such as 1-5 heteroatoms and 1-6 carbon atoms, such as 1-5 heteroatoms and 1-3 carbon atoms, in particular 5- or 6-membered rings with 1-4 heteroatoms or 1-2 heteroatoms selected from O, S and N, or optionally fused bicyclic rings with 1-4 heteroatoms, and wherein at least one ring is aromatic, e.g. pyridyl, quinolyl, isoquinolyl, indolyl, tetrazolyl, thiazolyl, imidazolyl, imidazo[1,2-a]pyrimidinyl, pyrazolyl, oxazolyl, oxadiazolyl, thiophenyl, 1,2,4-triazolyl, isoxazolyl, pyrrolidinyl, thienyl, pyrazinyl, pyrimidinyl, [1,2,3]triazolyl, isothiazolyl, tetrahydrofuranyl, imidazo[2,1-b]thiazolyl, benzimidazolyl, benzofuranyl, 2H-chromenyl, or benzofuranyl.

The term “heterocycloalkyl” is intended to indicate a cycloalkyl radical as defined above, including polycyclic radicals, optionally fused with carbocyclic rings, comprising 1-6 heteroatoms, preferably 1-3 heteroatoms, selected from O, N, or S, e.g. tetrahydropyranyl, morpholine, imidazolidinyl, benzo[1,3]dioxolyl, or piperidinyl.

The term “heterocycloalkenyl” is intended to indicate a cycloalkenyl radical as defined above, including polycyclic radicals, optionally fused with carbocyclic rings, comprising 1-6 heteroatoms, preferably 1-3 heteroatoms, selected from O, N, or S, e.g. 1,6-dihydropyridinyl, 2,3-dihydrobenzofuranyl, 4,5-dihydro-1H-[1,2,4]-triazolyl, 4,5-dihydro-oxazolyl, 1H-indazolyl, 1-H-pyrazolyl, or 4,5-dihydro-isoxazolyl.

The term “aryl” is intended to indicate a radical of aromatic carbocyclic rings comprising 6-20 carbon atoms, such as 6-14 carbon atoms, preferably 6-10 carbon atoms, in particular 5- or 6-membered rings, optionally fused carbocyclic rings with at least one aromatic ring, such as phenyl, naphthyl, anthracenyl, indenyl or indanyl.

The term “halogen” is intended to indicate a substituent form the 7^{th} main group of the periodic table, preferably fluoro, chloro and bromo.

The term “alkenylcarbonyloxy” is intended to indicate a radical of the formula —O—C(O)—R, wherein R is alkenyl as indicated above, e.g. acryloyloxy.

The term “amino” is intended to indicate a radical of the formula —NR₂, wherein each R independently represents hydrogen, alkyl, alkenyl, cycloalkyl, or aryl as indicate above, e.g. —NH₂, aminophenyl, methylamino, diethylamino, cyclohexylamino, —NH-phenyl, tert-butylamino or ethylamino.

The term “imino” is intended to indicate a radical of the formula ═N—R, wherein R represents hydrogen or alkyl as indicated above.

The term “alkoxy” is intended to indicate a radical of the formula —OR, wherein R is alkyl or alkenyl as indicated above, e.g. methoxy, ethoxy, n-propoxy, isopropoxy, butoxy, etc.

The term “alkylthio” is intended to indicate a radical of the formula —S—R, wherein R is alkyl as indicated above.

The term “alkoxycarbonyl” is intended to indicate a radical of the formula —C(O)—O—R, wherein R is alkyl as indicated above, e.g. methoxycarbonyl, ethoxycarbonyl, n-propoxycarbonyl, isopropoxycarbonyl, etc.

The term “alkylcarbonyloxy” is intended to indicate a radical of the formula —O—C(O)—R, wherein R is alkyl as indicated above, e.g. methylcarbonyloxy, or ethylcarbonyloxy.

The term “alkoxycarbonyloxy” is intended to indicate a radical of the formula —O—C(O)—O—R, wherein R is alkyl as indicated above.

The term “alkylcarbonyl” is intended to indicate a radical of the formula “—C(O)—R, wherein R is alkyl as indicated above, e.g. acetyl.

The term “alkylureido” is intended to indicate a radical of the formula “—NR′—C(O)—NH—R, wherein R' is hydrogen or alkyl as indicated above, and R is hydrogen, alkyl, or cycloalkyl as indicated above, e.g. —NH—C(O)—NH₂, methylureido, ethylureido, tert-butylureido, cyclohexylureido, methylthioureido, isopropylureido, or n-propylureido.

The term “alkylthioureido” is intended to indicate a radical of the formula “—NR′—C(S)—NH—R, wherein R′ is hydrogen or alkyl as indicated above, and R is hydrogen, alkyl, or cycloalkyl as indicated above, e.g. —NH—C(S)—NH₂.

The term “alkoxysulfonyloxy” is intended to represent a radical of the formula —O—S(O)₂—O—R, wherein R is alkyl as indicated above.

The term “aminosulfonyl” is intended to indicate a radical of the formula —S(O)₂—NR_{Z}, wherein each R independently represents hydrogen, alkyl or aryl as indicated above.

The term “aminocarbonyloxy” is intended to indicate a radical of the formula —NR′—C(O)—O—R, wherein R' is hydrogen or alkyl as indicated above, and R is alkyl as indicated above, e.g. aminocarbonyl-tert-butoxy.

The term “alkylsulfonylamino” is intended to indicate a radical of the formula —NR′—S(O)₂—R, wherein R is alkyl as indicated above, and R′ is hydrogen or alkyl as indicated above, e.g. methylsulfonylamino.

The term “arylsulfonylamino” is intended to indicate a radical of the formula —NR′—S(O)₂—R, wherein R is aryl as indicated above, and R′ is hydrogen or alkyl as indicated above, e.g. phenylsulfonylamino.

The term “heteroarylsulfonylamino” is intended to indicate a radical of the formula —NR′—S(O)₂—R, wherein R is heteroaryl as indicated above, and R′ is hydrogen or alkyl as indicated above, e.g. thiazolesulfonylamino.

The term “alkoxyimino” intended to indicate a radical of the formula ═N—O—R, wherein R is alkyl as indicated above, e.g. methoxyimino.

The term “aminocarbonyl” is intended to indicate a radical of the formula —C(O)—NR′₂, wherein each R′ is independently hydrogen, alkyl, alkenyl, or aryl as indicated above, e.g. carbamoyl, methylaminocarbonyl, ethylaminocarbonyl, propylaminocarbonyl, or butylaminocarbonyl.

The term “alkylcarbonylamino” is intended to indicate a radical of the formula —NR′—C(O)—R, wherein R′ is hydrogen or alkyl as indicated above, and R is alkyl as indicated above, e.g. acetylamino.

The term “heterocycloalkylcarbonylamino” is intended to indicate a radical of the formula —NR′—C(O)—R, wherein R′ is hydrogen or alkyl as indicated above, and R is heterocycloalkyl as indicated above, e.g. pyrrolidinylcarbonylamino.

The term “arylsulfonylamino” is intended to indicate a radical of the formula —NR′—S(O)₂—R, wherein R′ is hydrogen or alkyl as indicated above, and R is aryl as indicated above.

The term “arylsulfonyl” is intended to indicate a radical of the formula —S(O)₂—R, wherein R is aryl as indicated above.

The term “alkylsulfonyl” is intended to indicate a radical of the formula —S(O)₂—R, wherein R is alkyl as indicated above, e.g. methylsulfonyl.

The term “pharmaceutically acceptable salt” is intended to indicate salts prepared by reacting a compound of formula I with a suitable inorganic or organic acid, such as hydrochloric, hydrobromic, hydroiodic, sulfuric, nitric, phosphoric, formic, acetic, 2,2-dichloroaetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxy ethanesulfonic acid, toluenesulfonic, sulfamic or fumaric acid. Pharmaceutically acceptable salts of compounds of formula I may also be prepared by reaction with a suitable base such as sodium hydroxide, potassium hydroxide, magnesium hydroxide, calcium hydroxide, silver hydroxide, ammonia or the like.

The term “solvate” is intended to indicate a species formed by interaction between a compound, e.g. a compound of formula I, and a solvent, e.g. alcohol, glycerol or water, wherein said species are in a solid form. When water is the solvent, said species is referred to as a hydrate.

The term “Src” is used to indicate a protein tyrosine kinase of the Src family expressed in a wide range of cells and is inducibly expressed in macrophages. Src is involved in the signal transduction pathways of inflammatory gene expression, for instance mediating TNF-alpha expression in LPS stimulated macrophages.

The term “Yes” is used to indicate a protein tyrosine kinase of the Src family expressed in a wide range of cells. Yes is implicated in the signaling downstream of cytokine signaling in immune and inflammatory cells.

The term “Fyn” is used to indicate a protein tyrosine kinase of the Src family expressed in, i.a., T-cells, B-cells, NK cells and mast cells where it is involved in signaling via the T-cell receptor, adhesion mediated signaling. It has an essential role in mast cell degranulation and cytokine production.

The term “Lck” is used to indicate a protein tyrosine kinase of the Src family expressed in, i.a., T-cells and NK cells where it has a central role in T-cell activation and differentiation.

The term “Lyn” is used to indicate a protein tyrosine kinase of the Src family ubiquitously expressed in hematopoietic cells such as T-cells, B-cells, NK cells, neutrophils, eosinophils, macrophages, monocytes, mast cells and dendritic cells where it is involved, i.a., in modulation of B-cell responses.

The term “Hck” is used to indicate a protein tyrosine kinase of the Src family expressed in, i.a., neutrophils, eosinophils, monocytes, macrophages and dendritic cells where it is involved in transducing a variety of extracellular signals which ultimately affect cellular processes including proliferation, differentiation and migration.

The term “Fgr” is used to indicate a protein tyrosine kinase of the Src family expressed in, i.a., neutrophils, eosinophils, monocytes, macrophages and dendritic cells where it is involved in the signaling cascade from the B-cell receptor, FcR and the integrin family of receptors.

The term “Jak-2” is used to indicate a protein tyrosine kinase of the JAK (Janus protein tyrosine kinase) family highly expressed in immune cells where it is essential for signaling downstream of many cytokines and growth factors including the proinflammatory cytokines IL-6, IFN-γ, IL-3, IL-5 and GM-CSF.

The term “cKit” is used to indicate a receptor tyrosine kinase which is the receptor for stem cell factor (SCF) and is required for normal hematopoiesis. cKit plays an essential role in mast cell function as SCF is necessary for mast cell development, proliferation and survival. SCF is essential for optimal IgE/antigen-induced mast cell degranulation and cytokine production. Activation of c-kit induces eosinophil activation and degranulation.

The term “Fms/CSF-1R” is used to indicate a receptor tyrosine kinase which is the receptor for CSF-1 and is primarily expressed by monocytes and macrophages. CSF-1 plays a central role in macrophage effector functions during inflammation and regulates macrophage differentiation, survival and function.

The term “Raf-1” is used to indicate a tyrosine kinase-like serine/threonine kinase of the RAF family members of which are the main effectors recruited by GTP-bound Ras to activate the MEK-MAP kinase pathway. This pathway has been implicated in the expression of the proinflammatory cytokine GM-CSF and in the development of chronic inflammation by interfering with the longevity of neutrophils.

### Preferred Embodiments

Compounds of formula I and methods of preparing the compounds are disclosed in WO 2005/054179 which is hereby incorporated by reference in its entirety. In this publication, the compounds are indicated to be inhibitors of the receptor tyrosine kinase VEGF-R2 (KDR) which is involved in angiogenesis (formation of new blood vessels). Such inhibitors of VEGF-R2 have therefore been proposed for the treatment of diseases where angiogenesis plays an important role such as cancer, retinopathy and age-related macular degeneration. However, it has surprisingly been found that certain compounds previously disclosed in WO 2005/054179 as angiogenesis inhibitors are highly potent inhibitors of protein tyrosine kinases involved in inflammation and immune response and as such have potential as inhibitors of diseases where inflammation plays a major role in the pathogenesis, in particular inflammatory or autoimmune diseases involving Src family kinases.

In particular, compounds of formula I have been found to potently inhibit the activity of one or more of the protein tyrosine kinases Src, Yes, Fyn, Lyn, Fgr, Lck or Hck by at least 50%, or at least 60%, or at least 75%, or at least 80%, or at least 85%, or at least 90%, when tested at a concentration of 1 μM. Furthermore, compounds of formula I have been found to potently inhibit the protein tyrosine kinase Jak-2 by at least 50%, or at least 60%, or at least 75%, or at least 80%, or at least 85%, or at least 90%, when tested at a concentration of 1 μM. In addition, compounds of formula I have also been found to potently inhibit the serine/threonine kinase Raf-1 by at least 50%, or at least 60%, or at least 75%, or at least 80%, or at least 85%, or at least 90%, when tested at a concentration of 1 μM. In addition, compounds of formula I have also been found to potently inhibit the receptor tyrosine kinases cKit and Fsm/CSF-1R by at least 50%, or at least 60%, or at least 75%, or at least 80%, or at least 85%, or at least 90%, when tested at a concentration of 1 μM.

In a currently preferred embodiment, the compound of formula I is capable of inhibiting one or more of these kinases with an IC₅₀ of 200 nM or less as determined in a suitable in vitro assay such as the one disclosed in Example 1 below. Thus, the compound of formula I may be capable of inhibiting one or more of these kinases with an IC₅₀ of 100 nM or less, such as an IC₅₀ of 50 nM or less, in particular 30 nM or less, such as 25 nM or less, 20 nM or less, 15 nM or less or 10 nM or less. In a preferred embodiment, the compound of formula I is capable of inhibiting at least two, or at least 3, or at least 4, or at least 5, or at least 6, or at least 7, or at least 8, or at least 9, or at least 10, or preferably all of the kinases indicated above with an IC₅₀ of 200 nM or less.

It has surprisingly been found that compounds of formula I wherein R₂ comprises a functional group including a nitrogen atom such as an alkylureido, alkylthioureido, aminocarbonyl or aminosulfonyl group appear to be particularly effective inhibitors of Src family protein tyrosine kinases. Without being limited to any particular theory, it is believed that the presence of a nitrogen-containing functional group in the molecule affords specific ATP-competitive and/or non-competitive interactions at or near the active site of the kinase.

Examples of compounds of formula I which may be useful as antiinflammatory agents are
2-[(2-Amino-pyridin-4-ylmethyl-amino]-N-(4-cyano-benzyloxy)-benzamide (compound 1),
N-(4-Fluoro-benzyloxy)-2-[(2-morpholin-4-yl-pyridin-4-ylmethyl)-amino]-benzamide (compound 2),
N-Cyclopentylmethoxy-2-[(2-methanesulfonylamino-pyridin-4-ylmethyl-amino]-benzamide (compound 3),
N-(4-Cyano-benzyloxy)-2-[(2-methanesulfonylamino-pyridin-4-ylmethyl)-amino]-benzamide (compound 4),
N-(4-Cyano-benzyloxy)-2-{([2-(3-methyl-ureido)-pyridin-4-ylmethyl]-amino}-benzamide (compound 5),
N-(4-Cyano-2-methoxy-benzyloxy)-2-{[2-(3-methyl-ureido)-pyridin-4-ylmethyl]-amino}-benzamide (compound 6),
N-Cyclopentylmethoxy-2-{([2-(3-methyl-ureido)-pyridin-4-ylmethyl]-amino}-benzamide (compound 7),
N-(2,3-Difluoro-4-methyl-benzyloxy)-2-{[2-(3-methyl-ureido)-pyridin-4-ylmethyl]-amino}-benzamide (compound 8)
[3-(4-{[2-(4-Cyano-benzyloxycarbamoyl)-phenylamino]-methyl}-pyridin-2-yl)-ureido]-acetic acid ethyl ester (compound 9), (3-{4-[(2-Cyclopentylmethoxycarbamoyl-phenylamino)-methyl]-pyridin-2-yl}-ureido)-acetic acid ethyl ester (compound 10),
[3-(4-{([2-(4-Cyano-benzyloxycarbamoyl)-phenylamino]-methyl}-pyridin-2-yl)-ureido]-acetic acid (compound 11),
(3-{4-[(2-Cyclopentylmethoxycarbamoyl-phenylamino)-methyl]-pyridin-2-yl}-ureido)-acetic acid (compound 12),
2-Methyl-acrylic acid 2-[3-(4-{[2-(4-cyano-benzyloxycarbamoyl)-phenylamino]-methyl}-pyridin-2-yl)-ureido]-ethyl ester (compound 13),
2-Methyl-acrylic acid 2-(3-{4-[(2-cyclopentylmethoxycarbamoyl-phenylamino)-methyl]-pyridin-2-yl}-ureido)-ethyl ester (compound 14),
N-(4-Cyano-benzyloxy)-2-({2-[3-(2-hydroxy-ethyl)-ureido]-pyridin-4-ylmethyl}-amino)-benzamide (compound 15),
N-Cyclopentylmethoxy-2-({2-[3-(2-hydroxy-ethyl)-ureido]-pyridin-4-ylmethyl}-amino)-benzamide (compound 16),
Acetic acid (4-{[2-(4-cyano-benzyloxycarbamoyl)-phenylamino]-methyl}-pyridin-2-ylcarbamoyl)-methyl ester (compound 17),
Acetic acid {4-[(2-cyclopentylmethoxycarbamoyl-phenylamino)-methyl]-pyridin-2-ylcarbamoyl}-methyl ester (compound 18),
N-(4-Cyano-benzyloxy)-2-{[2-(2-hydroxy-acetylamino)-pyridin-4-ylmethyl]-amino}-benzamide (compound 19),
N-(4-Cyano-benzyloxy)-2-{[2-(cyclopropanecarbonyl-amino)-pyridin-4-ylmethyl]-amino}-benzamide (compound 20),
N-Cyclopentylmethoxy-2-{[2-(cyclopropanecarbonyl-amino)-pyridin-4-ylmethyl]-amino}-benzamide (compound 21),
N-Cyclopentylmethoxy-2-({2-[2-(2,5-dioxo-imidazolidin-4-yl)-acetylamino]-pyridin-4-ylmethyl}-amino)-benzamide (compound 22),
2-{[2-(3-Methyl-ureido)-pyridin-4-ylmethyl]-amino}-N-(tetrahydro-pyran-2-ylmethoxy)-benzamide (compound 23),
N-(4-Cyano-benzyloxy)-2-{[2-(3-isopropyl-ureido)-pyridin-4-ylmethyl]-amino}-benzamide (compound 24),
N-(4-Cyano-benzyloxy)-2-{[2-(3-ethyl-ureido)-pyridin-4-ylmethyl]-amino}-benzamide (compound 25),
N-Cyclopentylmethoxy-2-{([2-(3-isopropyl-ureido)-pyridin-4-ylmethyl]-amino}-benzamide (compound 26),
N-Cyclopentylmethoxy-2-{[2-(3-propyl-ureido)-pyridin-4-ylmethyl]-amino}-benzamide (compound 27),
N-Cyclopentylmethoxy-2-{[2-(3-ethyl-ureido)-pyridin-4-ylmethyl]-amino}-benzamide (compound 28),
N-Cyclopentylmethoxy-2-{[2-(3-methyl-thioureido)-pyridin-4-ylmethyl]-amino}-benzamide (compound 29),
2-{[2-(3-tert-Butyl-ureido)-pyridin-4-ylmethyl]-amino}-N-cyclopentylmethoxy-benzamide (compound 30),
N-(4-Cyano-benzyloxy)-2-{([2-(3-cyclohexyl-ureido)-pyridin-4-ylmethyl]-amino}-benzamide (compound 31),
2-{[2-(3-Cyclohexyl-ureido)-pyridin-4-ylmethyl]-amino}-N-cyclopentylmethoxy-benzamide (compound 32)
N-{4-[(2-Cyclopentylmethoxycarbamoyl-phenylamino)-methyl]-pyridin-2-yl}-isonicotinamide (compound 33),
1-(2,2,2-Trifluoro-acetyl)-pyrrolidine-2-carboxylic acid {4-[(2-cyclopentylmethoxycarbamoyl-phenylamino)-methyl]-pyridin-2-yl}-amide (compound 34),
1-(2,2,2-Trifluoro-acetyl)-pyrrolidine-2-carboxylic acid (4-{[2-(4-cyano-benzyloxycarbamoyl)-phenylamino]-methyl}-pyridin-2-yl)-amide (compound 35),
1-Acetyl-piperidine-4-carboxylic acid {4-[(2-cyclopentylmethoxycarbamoyl-phenylamino)-methyl]-pyridin-2-yl}-amide (compound 36),
1-Acetyl-piperidine-4-carboxylic acid (4-{[2-(4-cyano-benzyloxycarbamoyl)-phenylamino]-methyl}-pyridin-2-yl)-amide (compound 37),
Pyrrolidine-2-carboxylic acid (4-{[2-(4-cyano-benzyloxycarbamoyl)-phenylamino]-methyl}-pyridin-2-yl)-amide (compound 38), and
Pyrrolidine-2-carboxylic acid {4-[(2-cyclopentylmethoxycarbamoyl-phenylamino)-methyl]-pyridin-2-yl}-amide (compound 39).

A compound which is currently believed to be of particular interest for the present purpose is N-Cyclopentylmethoxy-2-{[2-(3-methyl-ureido)-pyridin-4-ylmethyl]-amino}-benzamide which is denoted Compound 6 in the examples below.

As a result of their inhibitory activity on Src family protein tyrosine kinases and other kinases, compounds of formula I are believed to be useful in the treatment of non-infectious inflammatory or autoimmune diseases or conditions in which protein tyrosine kinases of the Src family and/or the Jak-2 and/or Raf-1 and/or cKit and/or Fms/CSF-1R kinase are significantly involved.

Examples of such non-infectious inflammatory diseases or conditions are selected from the group consisting of acute inflammatory diseases such as acute lung injury, acute respiratory distress syndrome, allergy, anaphylaxis, sepsis or graft-vs-host disease, or chronic inflammatory diseases such as atopic dermatitis, Crohn's disease, ulcerative colitis, osteoarthritis, gout, psoriatic arthritis, hepatic cirrhosis or multiple sclerosis.

Examples of such autoimmune diseases are selected from the group consisting of autoimmune gastritis, Addison's disease, autoimmune hemolytic anemia, autoimmune thyroiditis, chronic idiopathic urticaria, chronic immune polynephropathy, diabetes, diabetic nephropathy, myasthenia gravis, pemphigus vulgaris, pernicious anemia, primary biliary cirrhosis, systemic lupus erythematosus and thyroid eye disease.

Compounds of formula I are currently believed to be particularly useful in the treatment of non-infectious inflammatory ocular diseases or conditions such as non-infectious (e.g. allergic) conjunctivitis, uveitis, iritis, keratitis, scleritis, episcleritis, sympathitic ophthalmitis, blepharitis, keratoconjunctivitis sicca, or immunological cornea graft rejection.

The dose in which the compound of formula I is administered to the patient will depend on the severity of the disease or condition to be treated and the route of administration. Generally, however, the compound of formula I will be administered in an amount of 0.1-500 mg/kg body weight.

The compound of formula I may suitably be incorporated in a pharmaceutical formulation in a form suitable for oral (including sustained or timed release), rectal, parenteral (including subcutaneous, intraperitoneal, intramuscular, intraarticular and intravenous), transdermal, ophthalmic, topical, nasal or buccal administration.

The formulations may conveniently be prepared by any of the methods well known in the art of pharmacy, e.g. as disclosed in Remington, The Science and Practice of Pharmacy, 20^{th} ed., 2000. All methods include the step of bringing the active ingredient into association with the carrier, which constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

Formulations suitable for oral administration and containing a compound of formula I may be in the form of discrete units as capsules, sachets, tablets or lozenges, each containing a predetermined amount of the active ingredient; in the form of a powder or granules; in the form of a solution or a suspension in an aqueous liquid or non-aqueous liquid, such as ethanol or glycerol; or in the form of an oil-in-water emulsion or a water-in-oil emulsion. Such oils may be edible oils, such as e.g. cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose, hydroxypropylmethylcellulose, hydroxypropylcellulose, carbomers and polyvinylpyrrolidone. The active ingredient may also be administered in the form of a bolus, electuary or paste.

A tablet may be made by compressing or moulding the active ingredient optionally with one or more accessory ingredients. Compressed tablets may be prepared by compressing, in a suitable machine, the active ingredient(s) in a free-flowing form such as a powder or granules, optionally mixed by a binder, such as e.g. lactose, glucose, starch, gelatine, acacia gum, tragacanth gum, sodium alginate, carboxymethylcellulose, methylcellulose, hydroxypropylmethylcellulose, polyethylene glycol, waxes or the like; a lubricant such as e.g. sodium oleate, sodium stearate, magnesium stearate, sodium benzoate, sodium acetate, sodium chloride or the like; a disintegrating agent such as e.g. starch, methylcellulose, agar, bentonite, croscarmellose sodium, sodium starch glycollate, crospovidone or the like or a dispersing agent, such as polysorbate 80. Moulded tablets may be made by moulding, in a suitable machine, a mixture of the powdered active ingredient and suitable carrier moistened with an inert liquid diluent.

Formulations for rectal administration may be in the form of suppositories in which the compound of the present invention is admixed with low melting water soluble or insoluble solids such as cocoa butter, hydrogenated vegetable oils, polyethylene glycol or fatty acids esters of polyethylene glycols, while elixirs may be prepared using myristyl palmitate.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. The formulation may be conveniently sterilised by for instance filtration through a bacteria retaining filter, addition of sterilising agent to the formulation, irradiation of the formulation or heating of the formulation. Liposomal formulations as disclosed in e.g. Encyclopedia of Pharmaceutical Technology, vol. 9, 1994, are also suitable for parenteral administration.

Alternatively, the compound of formula I may be presented as a sterile, solid preparation, e.g. a freeze-dried powder, which is readily dissolved in a sterile solvent immediately prior to use.

Transdermal formulations may be in the form of a plaster or a patch.

Formulations suitable for ophthalmic administration may be in the form of a sterile aqueous preparation of the active ingredients, which may be in microcrystalline form, for example, in the form of an aqueous microcrystalline suspension. Liposomal formulations or biodegradable polymer systems e.g. as disclosed in Encyclopedia of Pharmaceutical Technology, vol. 2, 1989, may also be used to present the active ingredient for ophthalmic administration.

Formulations suitable for topical or ophthalmic administration include liquid or semi-liquid preparations such as liniments, lotions, gels, applicants, oil-in-water or water-in-oil emulsions such as creams, ointments or pastes; or solutions or suspensions such as drops.

Formulations suitable for nasal or buccal administration include powder, self-propelling and spray formulations, such as aerosols and atomisers. Such formulations are disclosed in greater detail in e.g. Modern Pharmaceutics, 2^{nd} ed., G. S. Banker and C. T. Rhodes (Eds.), page 427-432, Marcel Dekker, New York; Modern Pharmaceutics, 3^{th} ed., G. S. Banker and C. T. Rhodes (Eds.), page 618-619 and 718-721, Marcel Dekker, New York and Encyclopedia of Pharmaceutical Technology vol. 10, 3 Swarbrick and J. C. Boylan (Eds), page 191-221, Marcel Dekker, New York

In addition to the aforementioned ingredients, the formulations of a compound of formula I may include one or more additional ingredients such as diluents, buffers, flavouring agents, colourant, surface active agents, thickeners, preservatives, e.g. methyl hydroxybenzoate (including anti-oxidants), emulsifying agents and the like.

When the active ingredient is administered in the form of salts with pharmaceutically acceptable non-toxic acids or bases, preferred salts are for instance easily water-soluble or slightly soluble in water, in order to obtain a particular and appropriate rate of absorption.

The invention is further described in the following examples which are not in any way intended to limit the scope of the invention as claimed.

### Example 1

### Kinase Screen

Compound 6 was tested in vitro at the concentration of 1 μM with 204 kinases from a screening panel including 70 tyrosine kinases (both receptor tyrosine kinases and protein tyrosine kinases) and 134 serine/threonine kinases. Each recombinant kinase (5-10 mU) was incubated with [γ-33P-ATP], protein substrate, compound, and the reaction initiated by the addition of MgATP. After incubation for 40 minutes, the reaction was stopped by the addition of 5 μL of a 3% phosphoric acid solution. 10 μL of the reaction was then spotted onto a P30 filtermat and washed three times for 5 minutes in 75 mM phosphoric acid and once in methanol prior to drying and scintillation counting. The final concentration of ATP in the assay was 10 μM.

Table 1 shows the tyrosine kinases involved in inflammation and immune response that Compound 6 inhibited by about 50% or more, together with the % inhibition obtained for each kinase and the IC₅₀ values, when available.

TABLE 1

| Group | Family | Name | % 1 μM | IC₅₀ nM |
|---|---|---|---|---|
| TK1 | Src | Src | 89; 87 | 65 |
| ″ | ″ | Yes | 98 | |
| ″ | ″ | Fyn | 91 | 92 |
| ″ | ″ | Fgr | 105 | 90 |
| ″ | ″ | Lck | 93 | 9 |
| ″ | ″ | Lyn | 100 | 4 |
| ″ | ″ | Hck | 100 | 47 |
| TK9 | JAK | Jak-2 | 90 | 148 |
| RTK3 | PDGFR | cKit | 91 | 23 |
| ″ | ″ | Fms/CSF-1R | 97 | 12 |

In addition Compound A was shown to inhibit the serine/threonine kinase Raf-1 (of the RAF family which is related to tyrosine kinases) by 91% at a concentration of 1 μM and an IC₅₀ of 14 nM.

The table shows potent in vitro effects of Compound 6 on tyrosine kinases with important roles in inflammation and immunity, including the activation and the function of T-cells, B-cells and macrophages. These kinases are potently inhibited with inhibition between 91 and 100% at 1 μM.

### Example 2

### LPS Induced TNFα Response

TNF-α is released primarily from monocytes/macrophages after stimulation with LPS both in vitro and in vivo. In the acute model “LPS induced TNF-α response” the ability of the test compound to inhibit the TNF-α release in vivo was measured in mice.

Compound 6 was dosed orally to C3H/HeN female mice (Taconic, Denmark), six mice per group, one hour prior to injecting LPS (1.0 mg LPS/kg i.p.). Blood samples were collected 80 to 90 minutes after the LPS administration. The plasma level of TNFα was analysed by sandwich ELISA.

Compound 6 showed a clear inhibition of TNFα in the mouse model. As seen in Table 2 below, the TNFα level was clearly inhibited both after i.p. and p.o. administration in doses from 3 to 30 mg/kg.

TABLE 2

| | | Compound 6 | | |
|---|---|---|---|---|
| Experiment | Adm. route | 3 mg/kg | 10 mg/kg | 30 mg/kg |
| LPS2405 | i.p. | | −51% | |
| LPS2505 | i.p. | −37% | −69% | −72% |
| LPS4405 | p.o. | −43% | −58% | −49% |
| | i.p. | | −75% | |
| LPS 4605 | i.p. | −52% | −40% | −48% |

### Example 3

### Graft Versus Host (GvH)

T-cell activation is a part of the acquired immune system and is an important mechanism of many inflammatory diseases. The local graft versus host (GvH) model is an in vivo model where a one way alloreactivity is induced: when purified lymphocytes from inbred Lewis rats are injected locally in the hind foot of Lewis×Brown rats, these respond with a localized T cell-dependent lymphoproliferative response to the allogeneic cells in the Lewis×Brown Norway hybrid rats.

Alloreactive lymphocytes from Lewis female rats (donor rats, 2×10⁷ cells in 100 μl) were injected subcutaneously into one of the hind paws to groups of 6-8 male LewBN hybrid rats (recipient rats) on day 0. After 7 days, the rats were euthanized and the local lymph node (In. popliteus (PLN)), the spleen and the thymus were excised and weighed. The test-compound was administrated to recipient rats once daily from day 0-6. When donor rats were treated, the test-compound was administrated once daily from day −7 to −1. The inhibition of weight increase of the PLNs was used to measure the effects of the compounds.

The model is used to evaluate the ability of the test compounds to inhibit T cell activation and/or to evaluate their general immunosuppressive effect.

Compound 6 was tested with different administration schedules: by p.o. administration day 0-6; by i.p. administration day 0-6; and by treatment of donor rats day −7 to −1.

After i.p. administration of Compound 6 (25 and 50 mg/kg) once daily from day 0-6 there was a pronounced inhibition of the lymph node size at 45-55% with only a small deviation both between the groups and among the rats in each group, no dose-response was observed.

The oral administration of 50 mg/kg once daily was tested in two experiments. Using this dosing schedule, some effects were observed; however, a large deviation was observed both among animals in the dosed groups and between the results obtained in two experiments.

Finally, the pre-treatment of the donor rats from day −7 to −1 with 25 mg/kg i.p. was tried both with and without the treatment of recipient rats. In the first experiment there was a pronounced inhibitory effect of the pre-treatment. However, this could not be reproduced in the second experiment.

TABLE 3

| | | Compound 6 | | | |
|---|---|---|---|---|---|
| Experiment | Administration | | | pretreat- | pre + |
| no. | route | 50 mg/kg | 25 mg/kg | ment | post |
| GvH0105 | p.o. | −71% | | | |
| GvH0205 | p.o. | −25% | | | |
| GvH0106 | i.p. | −52% | −46% | −41% | −44% |
| GvH0206 | i.p. | | −55% | 2% | −50% |

Results are stated as % of vehicle control.
Pretreatment: Recipients treated with methylcellulose day 0-6; Donors treated with Compound 6 i.p. day −7 to day −1
Pre + post (treatment): Recipients treated with Compound 6 i.p. day 0-6; Donors treated with Compound 6 i.p, day −7 to day −1

### Example 4

### Collagen Induced Arthritis (CIA)

Collagen induced arthritis is a commonly used and widely accepted arthritis model. Immunologically, the model is dominated by a Th1 response; it is antigen specific and depends on both the B- and T-cell response. This model is responsive to most therapies effective in rheumatoid arthritis in humans except NSAIDs.

### Materials

Collagen type II from chicken (Sigma C 9301)
0.05 M Acetic acid
Freunds complete adjuvant, 5 mg/ml (FCA5), SSI, Denmark
Freunds incomplete adjuvant (FIA), SSI, Denmark
Prednisolone (Prednisolonacetat Vet, 10 mg/ml (Intervet, Holland)
Suspension vehicle (4 g Tween-80, 2 g Carboxy-methyl cellulose 7H4XF, 8 g NaCl, 1 liter H₂O)

### Methods

### Mice

In order to induce arthritis, the mice were immunised intradermally (i.d.) at the base of the tail with 100 μl of CII emulsion (2 mg/ml) in FCA (2 mg/ml of M. tuberculosis). The mice were re-immunised i.d. 21 days later with 100 μl of CII emulsion (1 mg/ml) in FIA. The test compound was administrated once daily from the initiation of treatment to the day before the end of the experiment.

### Clinical Score for CIA

Starting from 21 days after the induction of CIA, the mice were weighed twice a week and the inflammation of the four paws was graded from 0 to 4:
0: Normal
1: Swelling of 1-2 toe joints or swelling of metatarsus/metacarpus without swelling of toe joints.
2: Swelling of >2 toe joints or swelling of toe joints and metatarsus/metacarpus
3: Severe swelling of the paw including metatarsus/metacarpus
4: Very severe swelling of the paw resulting in chapped skin. The animals are euthanised.
   The sum of the scores from the four paws is reported as the arthritis score for each mouse. The maximum score obtainable is therefore 16.

### Calculation of Treatment Effect

The area under curve (AUC) of the arthritis score was calculated for all mice and used as a disease index. The treatment effect (inhibitory effect) of the compounds is calculated as:

1−(AUC_{treatment group})/(AUC_{vehicle group})

### Statistics

The medians of the AUC from all groups were compared using the Kruskal-Wallis test. When P<0.05 in the Kruskal-Wallis test, the Mann Whitney test was used to compare the AUCs for individual mice in drug treated groups with the AUC for mice in the control group which were treated with vehicle (significance level P<0.05).

### Rats

Lewis inbred rats (Harlan), 6 females per group, approximately 11 weeks old were immunised intradermally (i.d.) at the base of the tail with 200 μl of CII emulsion (1 mg/ml) in incomplete Freunds adjuvant (FIA). The rats were re-immunised 10 days later with 50 μl of CII emulsion (1 mg/ml) in FIA. From day 12 the inflammation of each paw was graded and calculated as described for the mouse model. The test-compound was administrated orally from day 10 or from day 14. The last day of treatment was the day before the end of the experiment.

### Results

### Mice

In mice, the clinical symptoms of arthritis appear from day 21-30; however, the immunological process starts just after the first immunisation. Compound 6 inhibited the degree of arthritis significantly when the treatment was started before the first clinical signs of arthritis (day 10 or day 14) (Table 5). However, when the treatment was initiated after the first clinical signs of arthritis (day 21-28), there was no effect of the treatment with Compound 6 (Table 6).

TABLE 5

| | Compound: | | | |
|---|---|---|---|---|
| | Vehicle | | Compound 6 | Compound 6 |
| | day 10 | Prednisolone | day 10 | day 14 |
| Dose: | 0.1 ml/10 g | 3 mg/kg p.o. | 25 mg/kg | 25 mg/kg |
| AUC | | | | |
| Average | 167 | 27 | 95 | 105 |
| SD | 68 | 31 | 70 | 61 |
| | | P < 0.05 | P < 0.05 | P < 0.05 |
| Percent of | | −84% | −43% | −37% |
| vehicle control | | | | |
| (group 1) | | | | |

TABLE 6

| | Compound | | | |
|---|---|---|---|---|
| | Vehicle, | | Compound 6 | Compound 6 |
| | day 28 | Prednisolone | day 21 | day 28 |
| Dose: | 0.1 ml/10 g | 3 mg/kg p.o. | 25 mg/ml | 25 mg/ml |
| AUC | | | | |
| Average | 122 | 27 | 159 | 134 |
| SD | 75 | 31 | 58 | 52 |
| | | P < 0.05 | P = NS | P = NS |
| Percent of | | −78% | 30% | 9% |
| vehicle control | | | | |
| (group 1) | | | | |

### Rats

In the setup of the CIA rat model, the rats typically develop clinical signs of arthritis around day 14. Compound 6 was tested in two experiments; in the first experiment Compound 6 was tested at 50 mg/kg i.p. in two groups, and the treatment was initiated at day 10 and day 14, respectively. With the initiation of treatment on day 10, the signs of arthritis were completely inhibited, whereas when the treatment was initiated on day 14 there was no effect of Compound 6 (Table 7).

In the second experiment, Compound 6 was tested in 3 doses i.p. (5, 10 and 25 mg/kg) with treatment from day 10, and at the dose of 25 mg/kg with treatment from day 14. There was a dose dependent effect of Compound 6 when the treatment was initiated on day 10, resulting in a 79% inhibition of the score at the highest dose of 25 mg/kg, and resulting in 55% and 57% inhibition, respectively, at the dose of 5 and of 10 mg/kg. No significant effect was observed in the group which started dosing on day 14 (Table 8).

TABLE 7

| | Compound | | | | |
|---|---|---|---|---|---|
| | Methylcellulose | Prednisolon | Prednisolon | Comp. 6 | Comp. 6 |
| | Dose (mg/kg) | | | | |
| | 0.1 ml/100 g | 5 mg/kg p.o. | 5 mg/kg p.o. | 50, i.p. | 50, i.p. |
| Treated from | day 10 | day 10 | day 10 | day 10 | day 14 |
| Average | 36 | 0.2 | 9 | 0 | 40 |
| SD | 19 | 0.4 | 13 | 0 | 26 |
| | | P < 0.05 | P < 0.05 | P < 0.05 | P = NS |
| Percent of | | −100% | −74% | −100% | 13% |
| vehicle control | | | | | |
| (group 1) | | | | | |

TABLE 8

| | Compound | | | | | | |
|---|---|---|---|---|---|---|---|
| | Vehicle | Prednisolone | Prednisolone | Compound 6, day 10 i.p. | | | Comp. 6 i.p. |
| | Dose | | | | | | |
| | 0.1 ml/100 g | 5 mg/kg p.o. | 5 mg/kg p.o. | 5 mg/kg | 10 mg/kg | 25 mg/kg | 25 mg/kg |
| Treated from | day 10 | day 10 | day 14 | | | | day 14 |
| Average | 37 | 0 | 8 | 16 | 16 | 8 | 28 |
| SD | 24 | 0 | 14 | 14 | 10 | 10 | 19 |
| | | P < 0.05 | P < 0.05 | P < 0.05 | P = NS | P < 0.05 | P = NS |
| Percent of | | −−100% | −78% | −55% | −57% | −79% | −23% |
| vehicle control | | | | | | | |
| (group 1) | | | | | | | |

### Example 6

### Skin Inflammation Model

Compound 6 was tested in an acute oxazolone model of skin inflammation.

The acute oxazolone model is an allergic contact dermatitis model. In the model BALB/c mice are sensitised once to oxazolone by topical application of the chemical to the shaved abdomen seven days before challenge with oxazolone to the ear. The ear thickness is determined 24, 48, 72 and 96 hours post compound treatment and compared to treatment with vehicle. In this study mice were dosed with 100 mg/kg Compound 6 p.o. 30 min. post challenge and results from 72 hours show 28% inhibition of ear swelling compared to treatment with vehicle alone.

1. A compound of general formula I 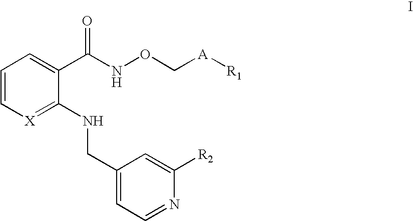
wherein X represents nitrogen or CH;
A represents a straight, branched and/or cyclic, saturated or unsaturated hydrocarbon radical, a heterocycloalkyl, a heterocycloalkenyl, or a heteroaryl, all of which are optionally substituted with one or more substituents independently selected from the group consisting of R₁;
R₁ represents oxo, halogen, trifluoromethyl, hydroxyl, amino, nitro, carboxy, cyano, alkoxy, alkylthio, alkoxycarbonyl, alkylcarbonyloxy, alkoxycarbonyloxy, alkylureido, alkylthioureido, alkylcarbonyl, alkoxysulfonyloxy, aminosulfonyl, arylsulfonyl, alkylsulfonylamino, arylsulfonylamino, heteroarylsulfonylamino, alkylsulfonyl, formyl, aminocarbonyl, alkylcarbonylamino, alkyl aminocarbonyl, amino carbonyloxy, heterocycloalkyl, heterocycloalkenyl, heteroaryl and a straight or branched, saturated or unsaturated hydrocarbon radical, wherein said amino, alkoxy, alkylthio, alkoxycarbonyl, alkylcarbonyloxy, alkoxycarbonyloxy, alkylureido, alkylthioureido, alkylcarbonyl, alkoxysulfonyloxy, aminosulfonyl, arylsulfonyl, alkylsulfonylamino, arylsulfonylamino, heteroarylsulfonylamino, alkylsulfonyl, aminocarbonyl, alkylcarbonylamino, alkylaminocarbonyl, aminocarbonyloxy, heterocycloalkyl, heterocycloalkenyl, heteroaryl and straight or branched, saturated or unsaturated hydrocarbon radical are optionally substituted by one or more substituents independently selected from the group consisting of R₃;
R₂ represents amino, aminosulfonyl, aminocarbonyl, alkylureido, alkylthioureido or aminocarbonyloxy, wherein each amino, aminosulfonyl, aminocarbonyl, alkylureido, alkylthioureido or aminocarbonyloxy is optionally substituted with one or more substituents independently selected from the group consisting of R₃;
R₃ represents hydrogen, cycloalkyl, alkyl, aryl, heteroaryl, heterocycloalkyl, heterocycloalkenyl, heterocycloalkyl-heteroaryl, heterocycloalkylcarbonylamino, cycloalkenyl, alkenyl, alkynyl, alkoxy, alkoxyimino, alkylthio, alkoxycarbonyl, alkylcarbonyloxy, alkenylcarbonyloxy, alkoxycarbonyloxy, alkylureido, alkylthioureido, alkylcarbonyl, alkoxysulfonyloxy, aminosulfonyl, alkylsulfonylamino, alkylsulfonyl, arylsulfonyl, formyl, aminocarbonyl, and alkylcarbonylamino, wherein said amino, imino, cycloalkyl, alkyl, aryl, heteroaryl, heterocycloalkyl, heterocycloalkenyl, heterocycloalkyl-heteroaryl, heterocycloalkylcarbonylamino, cycloalkenyl, alkenyl, alkynyl, alkoxy, alkoxyimino, alkylthio, alkoxycarbonyl, alkylcarbonyloxy, alkenylcarbonyloxy, alkoxycarbonyloxy, alkylureido, alkylthioureido, alkylcarbonyl, alkoxysulfonyloxy, aminosulfonyl, alkylsulfonylamino, alkylsulfonyl, arylsulfonyl, aminocarbonyl, and alkylcarbonylamino are optionally substituted by one or more substituents independently selected from the group consisting of hydrogen, halogen, oxo, thioxo, hydroxyl, amino, imino, nitro, carboxy, cyano, alkoxy, alkylthio, alkoxycarbonyl, alkylcarbonyloxy, alkoxycarbonyloxy, alkylcarbonyl, alkoxysulfonyloxy, aminosulfonyl, alkylsulfonylamino, alkylsulfonyl, arylsulfonyl, aminocarbonyloxy, heteroarylsulfonylamino, formyl, aminocarbonyl, trifluoromethyl, alkylcarbonylamino, heterocycloalkyl, heterocycloalkenyl, aryl, alkylureido, alkylthioureido, heteroaryl, cycloalkyl, alkyl, cycloalkenyl, alkenyl, alkynyl, and alkylaminocarbonyl;
and pharmaceutically acceptable salts, hydrates, or solvates thereof;
for use as an antiiflammatory agent capable of modulating the activity of a protein tyrosine kinase of the Src family of protein tyrosine kinases.

2. A compound according to claim 1 which is an inhibitor of at least one protein tyrosine kinase of the Src family involved in inflammation and/or immune response.

3. A compound according to claim 1, wherein the protein tyrosine kinase of the Src family is selected from Src, Yes, Fyn, Fgr, Lck, Lyn and Hck.

4. A compound according to claim 1, which is additionally an inhibitor of a protein tyrosine kinase of the JAK family, in particular JAK-2.

5. A compound according to claim 2 which is additionally an inhibitor of a serine/threonine kinase of the RAF family of serine/threonine kinases.

6. A compound according to claim 5, wherein the serine/threonine kinase of the RAF family is Raf-1.

7. A compound according to claim 1 which is additionally an inhibitor of a receptor tyrosine kinase selected from the group consisting of cKit and Fms/CSF-1R.

8. A compound according to claim 2 inhibiting said protein tyrosine kinase with an IC₅₀ of 200 nM or less.

9. A compound according to claim 8 inhibiting said protein tyrosine kinase with an IC₅₀ of 100 nM or less.

10. A compound according to claim 9 inhibiting said protein tyrosine kinase with an IC₅₀ of 50 nM or less, in particular 30 nM or less, such as 25 nM or less, 20 nM or less, 15 nM or less or 10 nM or less.

11. A compound according to claim 2 which is an inhibitor of at least two, or at least 3, or at least 4, or at least 5, or at least 6, or at least 7, or at least 8, or at least 9, or at least 10, or preferably all of the kinases listed in claim 2 with an IC₅₀ of 200 nM or less.

12. A compound according to claim 11, wherein R₂ is alkylureido, alkythioureido, aminocarbonyl or aminosulfonyl optionally substituted with one or more substituents independently selected from the group consisting of R₃ as defined in claim 1.

13. A compound according to claim 12 selected from the group consisting of
2-[(2-Amino-pyridin-4-ylmethyl)-amino]-N-(4-cyano-benzyloxy)-benzamide (compound 1),
N-(4-Fluoro-benzyloxy)-2-[(2-morpholin-4-yl-pyridin-4-ylmethyl)-amino]-benzamide (compound 2),
N-Cyclopentylmethoxy-2-[(2-methanesulfonylamino-pyridin-4-ylmethyl)-amino]-benzamide (compound 3),
N-(4-Cyano-benzyloxy)-2-[(2-methanesulfonylamino-pyridin-4-ylmethyl)-amino]-benzamide (compound 4),
N-(4-Cyano-benzyloxy)-2-{[2-(3-methyl-ureido)-pyridin-4-ylmethyl]-amino}-benzamide (compound 5),
N-(4-Cyano-2-methoxy-benzyloxy)-2-{[2-(3-methyl-ureido)-pyridin-4-ylmethyl]-amino}-benzamide (compound 6),
N-Cyclopentylmethoxy-2-{[2-(3-methyl-ureido)-pyridin-4-ylmethyl]-amino}-benzamide (compound 7),
N-(2,3-Difluoro-4-methyl-benzyloxy)-2-{[2-(3-methyl-ureido)-pyridin-4-ylmethyl]-amino}-benzamide (compound 8)
[3-(4-{[2-(4-Cyano-benzyloxycarbamoyl)-phenylamino]-methyl}-pyridin-2-yl)-ureido]-acetic acid ethyl ester (compound 9),
(3-{4-[(2-Cyclopentylmethoxycarbamoyl-phenylamino)-methyl]-pyridin-2-yl}-ureido)-acetic acid ethyl ester (compound 10),
[3-(4-{[2-(4-Cyano-benzyloxycarbamoyl)-phenylamino]-methyl}-pyridin-2-yl)-ureido]-acetic acid (compound 11),
(3-{4-[(2-Cyclopentylmethoxycarbamoyl-phenylamino)-methyl]-pyridin-2-yl}-ureido)-acetic acid (compound 12),
2-Methyl-acrylic acid 2-[3-(4-{[2-(4-cyano-benzyloxycarbamoyl)-phenylamino]-methyl}-pyridin-2-yl)-ureido]-ethyl ester (compound 13),
2-Methyl-acrylic acid 2-(3-{4-[(2-cyclopentylmethoxycarbamoyl-phenylamino)-methyl]-pyridin-2-yl}-ureido)-ethyl ester (compound 14),
N-(4-Cyano-benzyloxy)-2-({2-[3-(2-hydroxy-ethyl)-ureido]-pyridin-4-ylmethyl}-amino)-benzamide (compound 15),
N-Cyclopentylmethoxy-2-({2-[3-(2-hydroxy-ethyl)-ureido]-pyridin-4-ylmethyl}-amino)-benzamide (compound 16),
Acetic acid (4-{[2-(4-cyano-benzyloxycarbamoyl)-phenylamino]-methyl}-pyridin-2-ylcarbamoyl)-methyl ester (compound 17),
Acetic acid {4-[(2-cyclopentylmethoxycarbamoyl-phenylamino)-methyl]-pyridin-2-ylcarbamoyl}-methyl ester (compound 18),
N-(4-Cyano-benzyloxy)-2-{[2-(2-hydroxy-acetylamino)-pyridin-4-ylmethyl]-amino}-benzamide (compound 19),
N-(4-Cyano-benzyloxy)-2-{[2-(cyclopropanecarbonyl-amino)-pyridin-4-ylmethyl]-amino}-benzamide (compound 20),
N-Cyclopentylmethoxy-2-{[2-(cyclopropanecarbonyl-amino)-pyridin-4-ylmethyl]-amino}-benzamide (compound 21),
N-Cyclopentylmethoxy-2-({2-[2-(2,5-dioxo-imidazolidin-4-yl)-acetylamino]-pyridin-4-ylmethyl}-amino)-benzamide (compound 22),
2-{[2-(3-Methyl-ureido)-pyridin-4-ylmethyl]-amino}-N-(tetrahydro-pyran-2-ylmethoxy)-benzamide (compound 23),
N-(4-Cyano-benzyloxy)-2-{[2-(3-isopropyl-ureido)-pyridin-4-ylmethyl]-amino}-benzamide (compound 24),
N-(4-Cyano-benzyloxy)-2-{[2-(3-ethyl-ureido)-pyridin-4-ylmethyl]-amino}-benzamide (compound 25),
N-Cyclopentylmethoxy-2-{[2-(3-isopropyl-ureido)-pyridin-4-ylmethyl]-amino}-benzamide (compound 26),
N-Cyclopentylmethoxy-2-{[2-(3-propyl-ureido)-pyridin-4-ylmethyl]-amino}-benzamide (compound 27),
N-Cyclopentylmethoxy-2-{[2-(3-ethyl-ureido)-pyridin-4-ylmethyl]-amino}-benzamide (compound 28),
N-Cyclopentylmethoxy-2-{[2-(3-methyl-thioureido)-pyridin-4-ylmethyl]-amino}-benzamide (compound 29),
2-{[2-(3-tert-Butyl-ureido)-pyridin-4-ylmethyl]-amino}-N-cyclopentylmethoxy-benzamide (compound 30),
N-(4-Cyano-benzyloxy)-2-{[2-(3-cyclohexyl-ureido)-pyridin-4-ylmethyl]-amino}-benzamide (compound 31),
2-{[2-(3-Cyclohexyl-ureido)-pyridin-4-ylmethyl]-amino}-N-cyclopentylmethoxy-benzamide (compound 32)
N-{4-[(2-Cyclopentylmethoxycarbamoyl-phenylamino)-methyl]-pyridin-2-yl}-isonicotinamide (compound 33),
1-(2,2,2-Trifluoro-acetyl)-pyrrolidine-2-carboxylic acid {4-[(2-cyclopentylmethoxycarbamoyl-phenylamino)-methyl]-pyridin-2-yl}-amide (compound 34),
1-(2,2,2-Trifluoro-acetyl)-pyrrolidine-2-carboxylic acid (4-{[2-(4-cyano-benzyloxycarbamoyl)-phenylamino]-methyl}-pyridin-2-yl)-amide (compound 35),
1-Acetyl-piperidine-4-carboxylic acid {4-[(2-cyclopentylmethoxycarbamoyl-phenylamino)-methyl]-pyridin-2-yl}-amide (compound 36),
1-Acetyl-piperidine-4-carboxylic acid (4-{[2-(4-cyano-benzyloxycarbamoyl)-phenylamino]-methyl}-pyridin-2-yl)-amide (compound 37),
Pyrrolidine-2-carboxylic acid (4-{[2-(4-cyano-benzyloxycarbamoyl)-phenylamino]-methyl}-pyridin-2-yl)-amide (compound 38), and
Pyrrolidine-2-carboxylic acid {4-[(2-cyclopentylmethoxycarbamoyl-phenylamino)-methyl]-pyridin-2-yl}-amide (compound 39).

14. A compound of formula I as defined in claim 1 for use in the treatment of non-infectious inflammatory or autoimmune diseases or conditions in which protein tyrosine kinases of the Src family and/or the Jak-2 and/or Raf-1 and/or cKit and/or Fms/CSF-1R kinase are significantly involved.

15. A compound according to claim 14, wherein the non-infectious inflammatory disease or condition is selected from the group consisting of acute inflammatory diseases such as acute lung injury, acute respiratory distress syndrome, allergy, anaphylaxis, sepsis or graft-vs-host disease, or chronic inflammatory diseases such as atopic dermatitis, Crohn's disease, ulcerative colitis, osteoarthritis, gout, psoriatic arthritis, hepatic cirrhosis or multiple sclerosis.

16. A compound according to claim 14, wherein the autoimmune diseases is selected from the group consisting of autoimmune gastritis, Addison's disease, autoimmune hemolytic anemia, autoimmune thyroiditis, chronic idiopathic urticaria, chronic immune polynephropathy, diabetes, diabetic nephropathy, myasthenia gravis, pemphigus vulgaris, pernicious anemia, primary biliary cirrhosis, systemic lupus erythematosus and thyroid eye disease.

17. A compound according to claim 14, wherein the non-infectious inflammatory disease is a non-infectious inflammatory ocular disease or condition such as non-infectious (e.g. allergic) conjunctivitis, uveitis, iritis, keratitis, scleritis, episcleritis, sympathitic ophthalmitis, blepharitis, keratoconjunctivitis sicca, or immunological cornea graft rejection.

18. A method of modulating the activity of a protein tyrosine kinase of the Src family of protein tyrosine kinases involved in inflammation and/or immune response in cells, the method comprising contacting a cell expressing at least one protein tyrosine kinase of the Src family of protein tyrosine kinases with a compound of formula I as defined in claim 1 in an amount effective to modulate the activity of said protein tyrosine kinase in said cell.

19. A method according to claim 18, wherein the protein tyrosine kinase of the Src family is selected from Src, Yes, Fyn, Fgr, Lck, Lyn and Hck.

20. A method of modulating the activity of a protein tyrosine kinase of the JAK family of protein tyrosine kinases involved in inflammation and/or immune response in cells, the method comprising contacting a cell expressing at least one protein tyrosine kinase of the JAK family of protein tyrosine kinases with a compound of formula I as defined in claim 1 in an amount effective to modulate the activity of said protein tyrosine kinase in said cell.

21. A method according to claim 20, wherein the protein tyrosine kinase of the JAK family is JAK-2.

22. A method of modulating the activity of serine/threonine kinase of the RAF family involved in inflammation and/or immune response in cells, the method comprising contacting a cell expressing a RAF family kinase with a compound of formula I as defined in claim 1 in an amount effective to modulate the activity of said RAF family kinase in said cell.

23. A method according to claim 20, wherein the serine/threonine kinase of the RAF family is Raf-1.

24. A method of modulating the activity of a receptor tyrosine kinase selected from the group consisting of cKit and Fms/CSF-1R involved in inflammation and/or immune response in cells, the method comprising contacting a cell expressing at least one of cKit or Fms/CSF-1R with a compound of formula I as defined in claim 1 in an amount effective to modulate the activity of said receptor tyrosine kinase in said cell.

25. A method according to claim 18, wherein the compound of formula I is capable of inhibiting said protein tyrosine kinase with an IC₅₀ of 200 nM or less.

26. A method according to claim 25, wherein the compound of formula I is capable of inhibiting said protein tyrosine kinase with an IC₅₀ of 100 nM or less.

27. A method according to claim 26, wherein the compound of formula I is capable of inhibiting said protein tyrosine kinase with an IC₅₀ of 50 nM or less, in particular 30 nM or less, such as 25 nM or less, 20 nM or less, 15 nM or less or 10 nM or less.

28. A method according to claim 18, wherein the compound of formula I is capable of inhibiting at least two, or at least 3, or at least 4, or at least 5, or at least 6, or at least 7, or at least 8, or at least 9, or at least 10, or preferably all of the kinases indicated in claims 18-24 with an IC₅₀ of 200 nM or less.

29. A method according to claim 18, wherein R₂ is alkylureido, alkylthioureido, aminocarbonyl or aminosulfonyl optionally substituted with one or more substituents independently selected from the group consisting of R₃.

30. A method according to claim 29, wherein the compound of formula I is selected from the group consisting of
2-[(2-Amino-pyridin-4-ylmethyl)-amino]-N-(4-cyano-benzyloxy)-benzamide (compound 1),
N-(4-Fluoro-benzyloxy)-2-[(2-morpholin-4-yl-pyridin-4-ylmethyl)-amino]-benzamide (compound 2),
N-Cyclopentylmethoxy-2-[(2-methanesulfonylamino-pyridin-4-ylmethyl)-amino]-benzamide (compound 3),
N-(4-Cyano-benzyloxy)-2-[(2-methanesulfonylamino-pyridin-4-ylmethyl)-amino]-benzamide (compound 4),
N-(4-Cyano-benzyloxy)-2-{[2-(3-methyl-ureido)-pyridin-4-ylmethyl]-amino}-benzamide (compound 5),
N-(4-Cyano-2-methoxy-benzyloxy)-2-{[2-(3-methyl-ureido)-pyridin-4-ylmethyl]-amino}-benzamide (compound 6),
N-Cyclopentylmethoxy-2-{[2-(3-methyl-ureido)-pyridin-4-ylmethyl]-amino}-benzamide (compound 7),
N-(2,3-Difluoro-4-methyl-benzyloxy)-2-{[2-(3-methyl-ureido)-pyridin-4-ylmethyl]-amino}-benzamide (compound 8)
[3-(4-{[2-(4-Cyano-benzyloxycarbamoyl)-phenylamino]-methyl}-pyridin-2-yl)-ureido]-acetic acid ethyl ester (compound 9),
(3-{4-[(2-Cyclopentylmethoxycarbamoyl-phenylamino)-methyl]-pyridin-2-yl}-ureido)-acetic acid ethyl ester (compound 10),
[3-(4-{[2-(4-Cyano-benzyloxycarbamoyl)-phenylamino]-methyl}-pyridin-2-yl)-ureido]-acetic acid (compound 11),
(3-{4-[(2-Cyclopentylmethoxycarbamoyl-phenylamino)-methyl]-pyridin-2-yl}-ureido)-acetic acid (compound 12),
2-Methyl-acrylic acid 2-[3-(4-{[2-(4-cyano-benzyloxycarbamoyl)-phenylamino]-methyl}-pyridin-2-yl)-ureido]-ethyl ester (compound 13),
2-Methyl-acrylic acid 2-(3-{4-[(2-cyclopentylmethoxycarbamoyl-phenylamino)-methyl]-pyridin-2-yl}-ureido)-ethyl ester (compound 14),
N-(4-Cyano-benzyloxy)-2-({2-[3-(2-hydroxy-ethyl)-ureido]-pyridin-4-ylmethyl}-amino)-benzamide (compound 15),
N-Cyclopentylmethoxy-2-({2-[3-(2-hydroxy-ethyl)-ureido]-pyridin-4-ylmethyl}-amino)-benzamide (compound 16),
Acetic acid (4-{[2-(4-cyano-benzyloxycarbamoyl)-phenylamino]-methyl}-pyridin-2-ylcarbamoyl)-methyl ester (compound 17),
Acetic acid {4-[(2-cyclopentylmethoxycarbamoyl-phenylamino)-methyl]-pyridin-2-ylcarbamoyl}-methyl ester (compound 18),
N-(4-Cyano-benzyloxy)-2-{[2-(2-hydroxy-acetylamino)-pyridin-4-ylmethyl]-amino}-benzamide (compound 19),
N-(4-Cyano-benzyloxy)-2-{[2-(cyclopropanecarbonyl-amino)-pyridin-4-ylmethyl]-amino}-benzamide (compound 20),
N-Cyclopentylmethoxy-2-{[2-(cyclopropanecarbonyl-amino)-pyridin-4-ylmethyl]-amino}-benzamide (compound 21),
N-Cyclopentylmethoxy-2-({2-[2-(2,5-dioxo-imidazolidin-4-yl)-acetylamino]-pyridin-4-ylmethyl}-amino)-benzamide (compound 22),
2-{[2-(3-Methyl-ureido)-pyridin-4-ylmethyl]-amino}-N-(tetrahydro-pyran-2-ylmethoxy)-benzamide (compound 23),
N-(4-Cyano-benzyloxy)-2-{[2-(3-isopropyl-ureido)-pyridin-4-ylmethyl]-amino}-benzamide (compound 24),
N-(4-Cyano-benzyloxy)-2-{[2-(3-ethyl-ureido)-pyridin-4-ylmethyl]-amino}-benzamide (compound 25),
N-Cyclopentylmethoxy-2-{[2-(3-isopropyl-ureido)-pyridin-4-ylmethyl]-amino}-benzamide (compound 26),
N-Cyclopentylmethoxy-2-{[2-(3-propyl-ureido)-pyridin-4-ylmethyl]-amino}-benzamide (compound 27),
N-Cyclopentylmethoxy-2-{[2-(3-ethyl-ureido)-pyridin-4-ylmethyl]-amino}-benzamide (compound 28),
N-Cyclopentylmethoxy-2-{[2-(3-methyl-thioureido)-pyridin-4-ylmethyl]-amino}-benzamide (compound 29),
2-{[2-(3-tert-Butyl-ureido)-pyridin-4-ylmethyl]-amino}-N-cyclopentylmethoxy-benzamide (compound 30),
N-(4-Cyano-benzyloxy)-2-{[2-(3-cyclohexyl-ureido)-pyridin-4-ylmethyl]-amino}-benzamide (compound 31),
2-{[2-(3-Cyclohexyl-ureido)-pyridin-4-ylmethyl]-amino}-N-cyclopentylmethoxy-benzamide (compound 32)
N-{4-[(2-Cyclopentylmethoxycarbamoyl-phenylamino)-methyl]-pyridin-2-yl}-isonicotinamide (compound 33),
1-(2,2,2-Trifluoro-acetyl)-pyrrolidine-2-carboxylic acid {4-[(2-cyclopentylmethoxycarbamoyl-phenylamino)-methyl]-pyridin-2-yl}-amide (compound 34),
1-(2,2,2-Trifluoro-acetyl)-pyrrolidine-2-carboxylic acid (4-{[2-(4-cyano-benzyloxycarbamoyl)-phenylamino]-methyl}-pyridin-2-yl)-amide (compound 35),
1-Acetyl-piperidine-4-carboxylic acid {4-[(2-cyclopentylmethoxycarbamoyl-phenylamino)-methyl]-pyridin-2-yl}-amide (compound 36),
1-Acetyl-piperidine-4-carboxylic acid (4-{[2-(4-cyano-benzyloxycarbamoyl)-phenylamino]-methyl}-pyridin-2-yl)-amide (compound 37),
Pyrrolidine-2-carboxylic acid (4-{[2-(4-cyano-benzyloxycarbamoyl)-phenylamino]-methyl}-pyridin-2-yl)-amide (compound 38), and
Pyrrolidine-2-carboxylic acid {4-[(2-cyclopentylmethoxycarbamoyl-phenylamino)-methyl]-pyridin-2-yl}-amide (compound 39).

31. A method of reducing the proinflammatory activity in cells of a protein tyrosine kinases of the Src family of protein tyrosine kinases, the method comprising contacting a cell expressing at least one protein tyrosine kinase of the Src family with a compound of general formula I as defined in claim 1 in an amount effective to inhibit the activity of said protein tyrosine kinase in said cells.

32. A method according to claim 31, wherein the protein tyrosine kinase of the Src family is selected from Src, Yes, Fyn, Fgr, Lek, Lyn and Hck.

33. A method of reducing the proinflammatory activity in cells of a protein tyrosine kinase of the JAK family of protein tyrosine kinases, the method comprising contacting a cell expressing at least one protein tyrosine kinase of the JAK family of protein tyrosine kinases with a compound of formula I as defined in claim 1 in an amount effective to inhibit the activity of said protein tyrosine kinase in said cell.

34. A method according to claim 33, wherein the protein tyrosine kinase of the JAK-A family is JAK-2.

35. A method of reducing the proinflammatory activity in cells of serine/threonine kinase of the RAF family, the method comprising contacting a cell expressing a RAF family kinase with a compound of formula I as defined in claim 1 in an amount effective to inhibit the activity of said RAF family kinase in said cell.

36. A method according to claim 35, wherein the serine/threonine kinase of the RAF family is Raf-1.

37. A method of reducing the proinflammatory activity in cells of a receptor tyrosine kinase selected from the group consisting of cKit and Fms/CSF-1R, the method comprising contacting a cell expressing at least one of cKit or Fms/CSF-1R with a compound of formula I as defined in claim 1 in an amount effective to inhibit the activity of said receptor tyrosine kinase in said cell.

38. A method according to claim 31, wherein the compound of formula I is capable of inhibiting said protein tyrosine kinase with an IC₅₀ of 200 nM or less.

39. A method according to claim 38, wherein the compound of formula I is capable of inhibiting said protein tyrosine kinase with an IC₅₀ of 100 nM or less.

40. A method according to claim 39, wherein the compound of formula I is capable of inhibiting said protein tyrosine kinase with an IC₅₀ of 50 mM or less, in particular 30 mM or less, such as 25 nM or less, 20 nM or less, 15 nM or less or 10 nM or less.

41. A method according to claim 31, wherein the compound of formula I is capable of inhibiting at least two, or at least 3, or at least 4, or at least 5, or at least 6, or at least 7, or at least 8, or at least 9, or at least 10, or preferably all of the kinases listed in claims 28-34 with an IC₅₀ of 200 nM or less.

42. A method according to claim 41, wherein R₂ is alkylureido, alkylthioureido, aminocarbonyl or aminosulfonyl optionally substituted with one or more substituents independently selected from the group consisting of R₃.

43. A method according to claim 42, wherein the compound of formula I is selected from the group consisting of
2-[(2-Amino-pyridin-4-ylmethyl)-amino]-N44-cyano-benzyloxy)-benzamide (compound 1),
N-(4-Fluoro-benzyloxy)-2-[(2-morpholin-4-yl-pyridin-4-ylmethyl)-amino]-benzamide (compound 2),
N-Cyclopentylmethoxy-2-[(2-methanesulfonylamino-pyridin-4-ylmethyl)-amino]-benzamide (compound 3),
N44-Cyano-benzyloxy)-2-[(2-methanesulfonylamino-pyridin-4-ylmethyl)-amino]-benzamide (compound 4),
N44-Cyano-benzyloxy)-2-{[2-3-methyl-ureido)-pyridin-4-ylmethyl]-amino}-benzamide (compound 5),
N-(4-Cyano-2-methoxy-benzyloxy)-2-{[2-(3-methyl-ureido)-pyridin-4-ylmethyl]-amino}-benzamide (compound 6),
N-Cyclopentylmethoxy-2-{[2-(3-methyl-ureido)-pyridin-4-ylmethyl]-amino}-benzamide (compound 7),
N-(2,3-Difluoro-4-methyl-benzyloxy)-2-{[2-(3-methyl-ureido)-pyridin-4-ylmethyl]-amino}-benzamide (compound 8)
[3-(4-{[2-(4-Cyano-benzyloxycarbamoyl)-phenylamino]-methyl}-pyridin-2-yl)-ureido]-acetic acid ethyl ester (compound 9),
(3-{4-[(2-Cyclopentylmethoxycarbamoyl-phenylamino)-methyl]-pyridin-2-yl}-ureido)-acetic acid ethyl ester (compound 10),
[3-(4-{[2-(4-Cyano-benzyloxycarbamoyl)-phenylamino]-methyl}-pyridin-2-yl)-ureido]-acetic acid (compound 11),
(3-{4-[(2-Cyclopentylmethoxycarbamoyl-phenylamino)-methyl]-pyridin-2-yl}-ureido)-acetic acid (compound 12),
2-Methyl-acrylic acid 2-[3-(4-{[2-(4-cyano-benzyloxycarbamoyl)-phenylamino]-methyl}-pyridin-2-yl)-ureido]-ethyl ester (compound 13),
2-Methyl-acrylic acid 2-(3-{4-[(2-cyclopentyl-methoxycarbamoyl-phenylamino)-methyl]-pyridin-2-yl}-ureido)-ethyl ester (compound 14),
N-(4-Cyano-benzyloxy)-2-({2-[3-(2-hydroxy-ethyl)-ureido]-pyridin-4-ylmethyl}-amino)-benzamide (compound 15),
N-Cyclopentylmethoxy-2-({2-[3-(2-hydroxy-ethyl)-ureido]-pyridin-4-ylmethyl}-amino)-benzamide (compound 16),
Acetic acid (4-{[2-(4-cyano-benzyloxycarbamoyl)-phenylamino]-methyl}-pyridin-2-ylcarbamoyl)-methyl ester (compound 17),
Acetic acid {4-[(2-cyclopentylmethoxycarbamoyl-phenylamino)-methyl]-pyridin-2-ylcarbamoyl}-methyl ester (compound 18),
N-(4-Cyano-benzyloxy)-2-{[2-(2-hydroxy-acetylamino)-pyridin-4-ylmethyl]-amino}-benzamide (compound 19),
N-(4-Cyano-benzyloxy)-2-{[2-(cyclopropanecarbonyl-amino)-pyridin-4-ylmethyl]-amino}-benzamide (compound 20),
N-Cyclopentylmethoxy-2-{[2-(cyclopropanecarbonyl-amino)-pyridin-4-ylmethyl]amino}-benzamide (compound 21),
N-Cyclopentylmethoxy-2-({2-[2-(2,5-dioxo-imidazolidin-4-yl)-acetylamino]-pyridin-4-ylmethyl}-amino)-benzamide (compound 22),
2-{[2-(3-Methyl-ureido)-pyridin-4-ylmethyl]-amino}-N-(tetrahydro-pyran-2-ylmethoxy)-benzamide (compound 23),
N-(4-Cyano-benzyloxy)-2-{[2-(3-isopropyl-ureido)-pyridin-4-ylmethyl]-amino}-benzamide (compound 24),
N-(4-Cyano-benzyloxy)-2-{[2-(3-ethyl-ureido)-pyridin-4-ylmethyl]-amino}-benzamide (compound 25),
N-Cyclopentylmethoxy-2-{[2-(3-isopropyl-ureido)-pyridin-4-ylmethyl]-amino}-benzamide (compound 26),
N-Cyclopentylmethoxy-2-{[2-(3-propyl-ureido)-pyridin-4-ylmethyl]-amino}-benzamide (compound 27),
N-Cyclopentylmethoxy-2-{[2-(3-ethyl-ureido)-pyridin-4-ylmethyl]-amino}-benzamide (compound 28),
N-Cyclopentylmethoxy-2-{[2-(3-methyl-thioureido)-pyridin-4-ylmethyl]-amino}-benzamide (compound 29),
2-{[2-(3-tert-Butyl-ureido)-pyridin-4-ylmethyl]-amino}-N-cyclopentylmethoxy-benzamide (compound 30),
N-(4-Cyano-benzyloxy)-2-{[2-(3-cyclohexyl-ureido)-pyridin-4-ylmethyl]-amino}-benzamide (compound 31),
2-{[2-(3-Cyclohexyl-ureido)-pyridin-4-ylmethyl]-amino}-N-cyclopentylmethoxy-benzamide (compound 32)
N-{4-[(2-Cyclopentylmethoxycarbamoyl-phenylamino)-methyl]-pyridin-2-yl}-isonicotinamide (compound 33),
1-(2,2,2-Trifluoro-acetyl)-pyrrolidine-2-carboxylic acid {4-[(2-cyclopentylmethoxycarbamoyl-phenylamino)-methyl]-pyridin-2-yl}-amide (compound 34),
1-(2,2,2-Trifluoro-acetyl)-pyrrolidine-2-carboxylic acid (4-{[2-(4-cyano-benzyloxycarbamoyl)-phenylamino]-methyl}-pyridin-2-yl)-amide (compound 35),
1-Acetyl-piperidine-4-carboxylic acid {4-[(2-cyclopentylmethoxycarbamoyl-phenylamino)-methyl]-pyridin-2-yl}-amide (compound 36),
1-Acetyl-piperidine-4-carboxylic acid (4-{[2-(4-cyano-benzyloxycarbamoyl)-phenylamino]-methyl}-pyridin-2-yl)-amide (compound 37),
Pyrrolidine-2-carboxylic acid (4-{[2-(4-cyano-benzyloxycarbamoyl)-phenylamino]-methyl}-pyridin-2-yl)-amide (compound 38), and
Pyrrolidine-2-carboxylic acid {4-[(2-cyclopentylmethoxycarbamoyl-phenylamino)-methyl]-pyridin-2-yl}-amide (compound 39).

44. Use of a compound of general formula I as defined in claim 1 for the preparation of a pharmaceutical composition for the prevention or treatment of a non-infectious inflammatory or autoimmune disease or condition in which at least one protein tyrosine kinase of the Src family of protein tyrosine kinases and/or the Jak-2 and/or Raf-1 and/or cKit and/or Fms/CSF-1R kinase are significantly involved.

45. The use according to claim 44, wherein the non-infectious inflammatory disease or condition is selected from the group consisting of acute inflammatory diseases such as acute lung injury, acute respiratory distress syndrome, allergy, anaphylaxis, sepsis or graft-vs-host disease, or chronic inflammatory diseases such as allergy, anaphylaxis, atopic dermatitis, Crohn's disease, ulcerative colitis, osteoarthritis, gout, psoriatic arthritis, hepatic cirrhosis or multiple sclerosis.

46. The use according to claim 44, wherein the autoimmune diseases is selected from the group consisting of autoimmune gastritis, Addison's disease, autoimmune hemolytic anemia, autoimmune thyroiditis, chronic idiopathic urticaria, chronic immune polynephropathy, diabetes, diabetic nephropathy, myasthenia gravis, pemphigus vulgaris, pernicious anemia, primary biliary cirrhosis, systemic lupus erythematosus and thyroid eye disease.

47. The use according to claim 44, wherein the non-infectious inflammatory disease is a non-infectious inflammatory ocular disease or condition such as non-infectious (e.g. allergic) conjunctivitis, uveitis, iritis, keratitis, scleritis, episcleritis, sympathitic ophthalmitis, blepharitis, keratoconjunctivitis sicca, or immunological cornea graft rejection.

48. A method of preventing or treating a non-infectious inflammatory or autoimmune disease or condition in which at least one protein tyrosine kinase of the Src family of protein tyrosine kinases and/or the Jak-2 and/or Raf-1 and/or cKit and/or Fms/CSF-1R kinase are significantly involved, the method comprising administering, to a patient in need thereof, an effective amount of a compound of general formula I as defined in claim 1.

49. The method of claim 48, wherein the non-infectious inflammatory disease or condition is selected from the group consisting of acute inflammatory diseases such as acute lung injury, acute respiratory distress syndrome, allergy, anaphylaxis, sepsis or graft-vs-host disease, or chronic inflammatory diseases such as atopic dermatitis, Crohn's disease, ulcerative colitis, osteoarthritis, gout, psoriatic arthritis, hepatic cirrhosis or multiple sclerosis.

50. The method of claim 48, wherein the autoimmune diseases is selected from the group consisting of autoimmune gastritis, Addison's disease, autoimmune hemolytic anemia, autoimmune thyroiditis, chronic idiopathic urticaria, chronic immune polynephropathy, diabetes, diabetic nephropathy, myasthenia gravis, pemphigus vulgaris, pernicious anemia, primary biliary cirrhosis, systemic lupus erythematosus and thyroid eye disease.

51. The method of claim 48 wherein the non-infectious inflammatory disease is a non-infectious inflammatory ocular disease or condition such as non-infectious (e.g. allergic) conjunctivitis, uveitis, iritis, keratitis, scleritis, episcleritis, sympathitic ophthalmitis, blepharitis, keratoconjunctivitis sicca, or immunological cornea graft rejection.
